(19) United States
(12) Patent Application Publication
(10) Pub. No.: US 2013/0325015 A1
(43) Pub. Date: Dec. 5, 2013

(54) Surgical sliding shaft instrument

(71) Applicant: S. u. A. Martin GmbH & Co. KG, Rietheim-Weilheim (DE)

(72) Inventor: Martin Grasshoff, Rietheim-Weilheim (DE)

(21) Appl. No.: 13/907,386

(22) Filed: May 31, 2013

(30) Foreign Application Priority Data
May 31, 2012 (DE) 10 2012 209 247.1

(51) Int. Cl. A61B 17/16

(52) U.S. Cl. CPC A61B 17/1611
USPC 606/83

(57) ABSTRACT

A surgical sliding shaft instrument is provided. The instrument can include a shaft part, a slide part, which is guided so as to be movable in a longitudinal direction on the shaft part, working elements disposed on distal ends of the shaft part and the slide part, and a proximal handle for reciprocal movement between a rest position and an operative position. The shaft part and the slide part can abut one another with sliding surfaces. The slide part can be interrupted by first rinsing slots. The shaft part can be interrupted by second rinsing slots. The first rinsing slots of the slide part and the second rinsing slots of the shaft part can be constructed and disposed so that in the rest position of the instrument the first rinsing slots and the second rinsing slots adjoin one another in the longitudinal direction alternately without overlapping one another.

### CROSS REFERENCE TO RELATED APPLICATIONS

This application claims priority to German Patent Application No. DE 10 2012 209 247.1 filed May 31, 2012, which is hereby incorporated by reference in its entirety.

### DESCRIPTION

The application relates to a surgical sliding shaft instrument.

Sliding shaft instruments are used in surgery for the most varied purposes and in the most varied designs. A feature common to these sliding shaft instruments is a shaft which consists of a shaft part and a slide part which is guided so as to be longitudinally movable on this shaft part. Working elements which co-operate for the surgical intervention are disposed at the distal end of the shaft part and of the slide part. A handle is disposed on the proximal end of the shaft and serves to move the slide part and the shaft part in the longitudinal direction against one another in order to move them out of a rest position into an active position in which the working elements come into engagement.

In such sliding shaft instruments, the shaft part and the slide part are in contact with one another by sliding surfaces. When the sliding shaft instrument is in use, not only the distal working elements come into contact with the actual operating area. Contaminants, for example tissue particles, wound exudate, blood or the like, can also enter the region of the sliding surfaces between the shaft part and the slide part. Therefore when cleaning and sterilizing the instrument it is important also to clean the sliding surfaces of the slide part and the shaft part which abut one another. For this purpose in a sliding shaft instrument known from DE 20 2009 001 811 U1 a continuous cleaning gap is provided over the entire width between the mutually facing surfaces of the slide part and the shaft part. Rinsing slots which extend in the longitudinal direction of the slide part, are distributed over the longitudinal extent thereof and lead from the upper face of the slide part to the sliding surface, lead into said cleaning gap. Only a few such rinsing slots are provided at a relatively large reciprocal spacing in the longitudinal direction, since the actual rinsing takes place through the laterally open cleaning gap. The laterally open cleaning gap itself favors the penetration of contaminants and impairs the appearance of the sliding shaft instrument.

One object is to create a sliding shaft instrument which also enables a reliable cleaning and sterilization of the sliding surfaces between the slide part and the shaft part, wherein the sliding surfaces of the slide part and the shaft part abut one another.

### SUMMARY

These objects are achieved by a surgical sliding shaft instrument with the structures and features described herein.

In the sliding shaft instrument the shaft part and the slide part which is movably guided thereon abut one another with respective sliding surfaces. First rinsing slots in the slide part and second rinsing slots in the shaft part serve for rinsing of the sliding surfaces. The first rinsing slots extend from the upper face of the slide part through this slide part to the sliding surface. The second rinsing slots extend from the lower face of the shaft part through this shaft part to the sliding surface. In this case the first and second rinsing slots are distributed in the longitudinal direction of the shaft and are configured so that the first rinsing slots and the second rinsing slots adjoin one another in the longitudinal direction of the shaft, alternately without gaps or overlapping one another, when the instrument is in its rest position. This arrangement of the rinsing slots in the slide part and in the shaft part ensures that over the entire longitudinal extent of the sliding surfaces which abut one another are rinsed uninterruptedly through the rinsing slots leading alternately through the slide part and through the shaft part when the instrument is cleaned in its rest position. The rinsing slots lead from the upper face of the slide part or from the lower face of the shaft part to the respective sliding surfaces which abut one another, so that these sliding surfaces abut one another on the two lateral outer faces of the shaft without a gap. As a result lateral penetration of contaminants between the slide part and the shaft part are kept to a minimum. The slide part may be guided over the entire length in sliding abutment on the shaft part. The sliding shaft instrument offers an attractive appearance in side view.

The rinsing slots also enable cleaning and sterilization of the sliding surfaces of the slide part and the shaft part which one another, so that dismantling of the instrument for cleaning and sterilization is not necessary. If the handle of the sliding shaft instrument is constructed with pivotable handle branches, then the cleaning and sterilization can be further improved by additional rinsing bores and/or rinsing channels leading into the end region of the handle in which the two handle branches are connected to one another and one handle branch engages on the slide part.

### BRIEF DESCRIPTION OF DRAWINGS

A sliding shaft instrument is explained in greater detail below with reference to an embodiment which is illustrated in the drawings. In the drawings:

FIG. 1 shows a side view of a sliding shaft instrument, wherein the shaft is sectioned vertically in the longitudinal direction,

FIG. 2 shows an enlarged vertical cross-section of the shaft according to the section line A-B in FIG. 1,

FIG. 3 shows a representation corresponding to FIG. 1, wherein further rinsing options are illustrated,

FIG. 4 shows a plan view of the proximal side of the pivotable handle branch, and

FIG. 5 shows a plan view of the pivotable handle branch.

### DETAILED DESCRIPTION

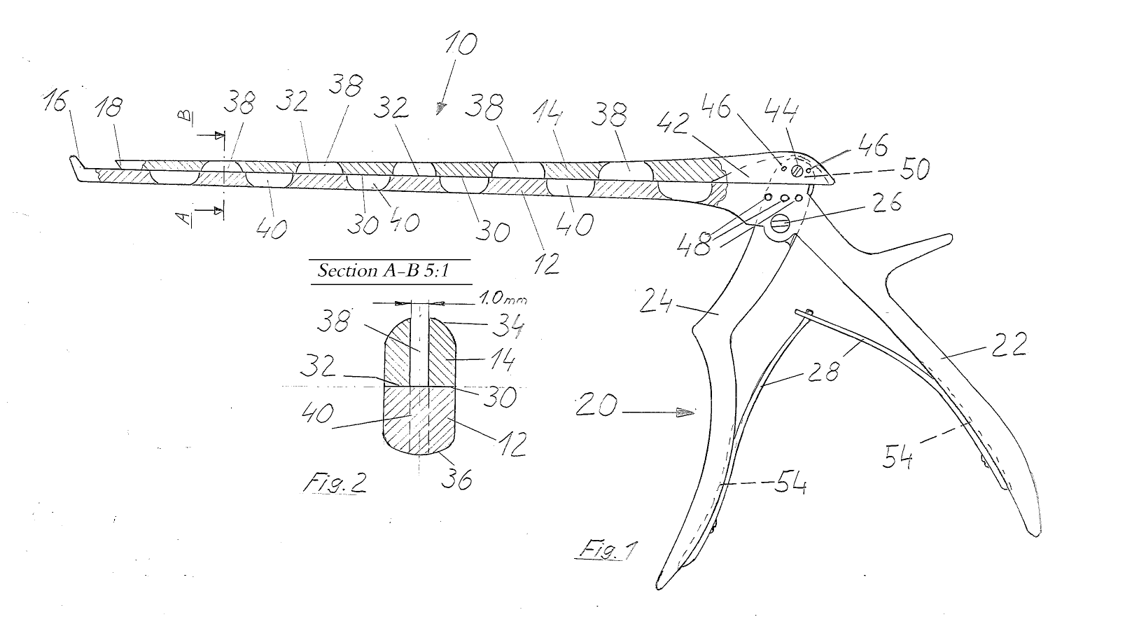

In the drawings, an embodiment is illustrated in which the sliding shaft instrument is configured as a punch. The sliding shaft instrument has an elongate shaft 10 which consists of a lower shaft part 12 and an upper slide part 14. The slide part 14 is guided so as to be longitudinally movable on the shaft part 12. Respective working elements are disposed on the distal ends of the shaft part 12 and of the slide part 14. In the illustrated embodiment of a punch, a punch foot 16 is disposed for example at the distal end of the shaft part 12, whilst a punch cutter blade 18 is constructed at the distal end of the slide part 14. In the rest position of the sliding shaft instrument illustrated in FIG. 1 the punch cutter blade 18 is remote from by the punch foot 16. If the slide part 14 is moved in the distal direction out of the rest position shown in FIG. 1 into an operative position, then acts the punch cutter blade 18 co-operates with the punch foot 16 in order to carry out a surgical punching operation. Other forms of working elements are likewise possible in the context of the disclosure.

A handle 20 is disposed at the proximal end of the shaft 10. The handle 20 serves for moving the slide part 14 in the longitudinal direction relative to the shaft part 12. In particular the slide part 14 is moved by means of the handle 20 from the rest position shown in FIG. 1 into the operative position.

In the illustrated embodiment the handle 20 has a fixed first handle branch 22 which is rigidly and integrally connected to the shaft part 12 and is angled relative to the longitudinal axis of the shaft. A second handle branch 24 is connected pivotably about a pivot joint 26 to the first handle branch 22. In the region of the pivot joint 26, the second handle branch 24 is received with a tab 50 in a receiving slot of the first handle branch 22. The tab 50 is extended beyond the pivot joint 26 and engages with this extended tab on the proximal end of the slide part 14. Springs 28 inserted between the handle branches 22 and 24 spread the handle branches 22 and 24 apart, so that the second handle branch 24 draws the slide part 14 in the proximal direction into the rest position shown in FIG. 1. If the handle branches 22 and 24 are compressed against the force of the springs 28, the second handle branch 24 pushes the slide part 14 in the distal direction into the operative position. Other forms of the handle 20 are likewise possible in the context of the disclosure. For example the handle may be disposed axially with respect to the shaft.

In this respect the sliding shaft instrument is of conventional construction. Other designs of the sliding shaft instrument are known and familiar to the person skilled in the art from the prior art. The structure and functionality explained below can also be used in such other designs of the sliding shaft instrument.

According to the disclosure the slide part 14 is guided so as to be longitudinally movable on the shaft part 12. This guiding can be carried out in a manner which is known per se and is therefore not shown in greater detail in the drawings.

The slide part 14 abuts an upper sliding surface 32 of the shaft part 12 with a lower sliding surface 30. The sliding surfaces 30 and 32 are in sliding contact with one another over the entire length of the shaft 10 and over the width thereof. As FIG. 2 shows, the shaft part 12 and the slide part 14 each have a substantially rectangular cross-section, wherein the sliding surfaces 30 and 32 form the narrow sides of this rectangular cross-section which abut one another. The upper face 34 of the slide part and the lower face 36 of the shaft part are each rounded. The width of the shaft part 12 and the slide part 14 and thus the width of the sliding surfaces 30 and 32 which abut one another is for example approximately 4 mm.

In the slide part 14 a plurality of first rinsing slots 38 are distributed in the longitudinal direction of the shaft 10. The rinsing slots 38 are elongate in the longitudinal direction of the slide part 14 and lead from the upper face 34 through the slide part 14 to the sliding surface 30. The rinsing slots 38 have the same width throughout, i.e. they are delimited by plane-parallel side faces. As FIG. 2 shows, the rinsing slots 38 are disposed centrally in relation to the width of the slide part 14. The scope and the length of the rinsing slots 38 may be freely chosen per se in order to link an optimal rinsing with the mechanical functionality of the instrument. The width of the rinsing slot 38 is for example approximately a quarter of the width of the slide part. For example the width the rinsing slots is approximately 0.5 to 1.3 mm, preferably approximately 1.0 mm. The length the rinsing slots 38 in the longitudinal direction is approximately 10 to 15 mm. The rinsing slots 38 are distributed in the longitudinal direction of the slide part 14 so that their reciprocal spacing in the longitudinal direction is substantially equal to their length in each case. As can be seen from FIG. 1, the first rinsing slots 38 are milled into the slide part 14, preferably starting from the sliding surface 30, so that the distal and proximal edges of the rinsing slots 38 extend in the shape of an arc of a circle and the length of the rinsing slots 38 reduces from the sliding surface 30 towards the upper face 34.

Second rinsing slots 40 are correspondingly constructed in the shaft part 12. The second rinsing slots 40 have substantially the same shape as the first rinsing slots 38 of the slide part 14. The second rinsing slots 40 lead due to the shaft part 12 from the lower face 36 to the sliding surface 32. The length and the width of the second rinsing slots 40 correspond substantially to the length and the width of the first rinsing slots 38. Also the reciprocal spacing of the second rinsing slots 40 in the longitudinal direction of the shaft 10 corresponds to the length of the second rinsing slots 40. The second rinsing slots 40 are milled into the shaft part 12, preferably starting from the sliding surface 32, so that the length of the second rinsing slots 40 reduces from the sliding surface 32 towards the lower face 36.

The arrangement of the first rinsing slots 38 in the slide part 14 and the second rinsing slots 40 in the shaft part 12 can be seen in FIG. 1. If the sliding shaft instrument is located in the rest position shown in FIG. 1, in which the handle 20 is not actuated, then the handle branches 22 and 24 are spread apart by the springs 28 and the slide part 14 is drawn by the pivotable second handle branch 24 into its proximal end position. In this rest position the first rinsing slots 38 of the slide part 14 are each aligned in the longitudinal direction with the longitudinal section of the sliding surface 32 of the shaft part 12 between the second rinsing slots 40. Conversely the second rinsing slots 40 of the slide part 12 are each aligned with the longitudinal sections of the sliding surface 30 of the slide part 14 between the first rinsing slots 38. Thus the first rinsing slots 38 of the slide part 14 and the second rinsing slots 40 of the shaft part 12 adjoin one another from above and from below alternately in the longitudinal direction substantially without gaps. A slight overlap of the rinsing slots 38 and 40 in longitudinal direction is of course also possible.

If the sliding shaft instrument is cleaned then it is located in the rest position shown in FIG. 1. Rinsing liquid can penetrate through the first rinsing slots 38 and the second rinsing slots 40. In the region of the first rinsing slots 38 the rinsing liquid penetrating through the first rinsing slots 38 cleans the sliding surface 32 of the shaft part 12 above the entire width and the sliding surface 30 of the slide part 14 in the regions on both sides of the rinsing slot 38. Conversely, in the longitudinal region of the second rinsing slots 40 the rinsing liquid penetrating into the first rinsing slots 40 cleans the sliding surface 30 of the slide part 14 above the entire width thereof and the sliding surface 32 of the shaft part 12 on both sides of the rinsing slots 40. In this way the sliding surfaces 30 and 32 are rinsed completely over the entire length of the shaft 10. In each region of the length of the sliding surface 30 and 32 the rinsing liquid only has to flow through at most a width of the one another adjoining sliding surfaces 30 and 32 which corresponds to the wall thickness of the shaft part 12 or of the slide part 14 to the side of the respective rinsing slot 40 or 38. Thus a complete and thorough rinsing and cleaning of the entire sliding surfaces 30 and 32 is ensured, without the slide part 14 having to be removed from the shaft part 12, i.e. without the sliding shaft instrument having to be dismantled for cleaning.

In the embodiment illustrated in the drawings, in which the handle 20 is formed by a fixed first handle branch 22 and a pivotable second handle branch 24 which project laterally from the shaft 10, an additional rinsing and cleaning in the end region of the instrument is possible. In this case the part of the instrument in which in the region of the pivot joint 26 the pivotable second handle branch 24 with its tab 50 is received in the receiving slot of the fixed handle branch 22 and with this tab 50 extended beyond the pivot joint 26 engages on the slide part 14, is designated as the end region. In this end region a recess 42 is milled into the lower face of the slide part 14 through which a transverse driver pin 44 or a driver screw 44 pass. The extended tab 50 of the pivotable second handle branch 24 surrounds this transverse driver pin 44 so that when the handle branch 24 is pivoted the slide part 14 is moved in the longitudinal direction on the shaft part 12. Rinsing bores 46 in this end region lead laterally through the wall of the slide part 14 into the recess 42. Further rinsing bores 48 lead through the wall of the shaft part 12 into the receiving slot for the tab 50 of the second handle branch 24. Cleaning and rinsing of the end region is also possible through the rinsing bores 46 and 48 without the sliding shaft instrument having to be dismantled.

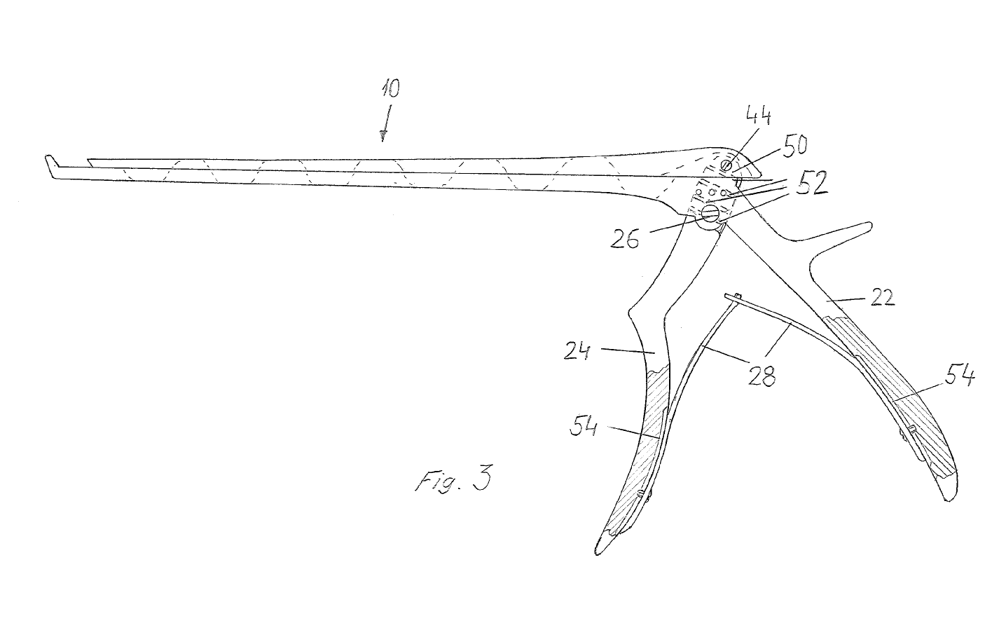

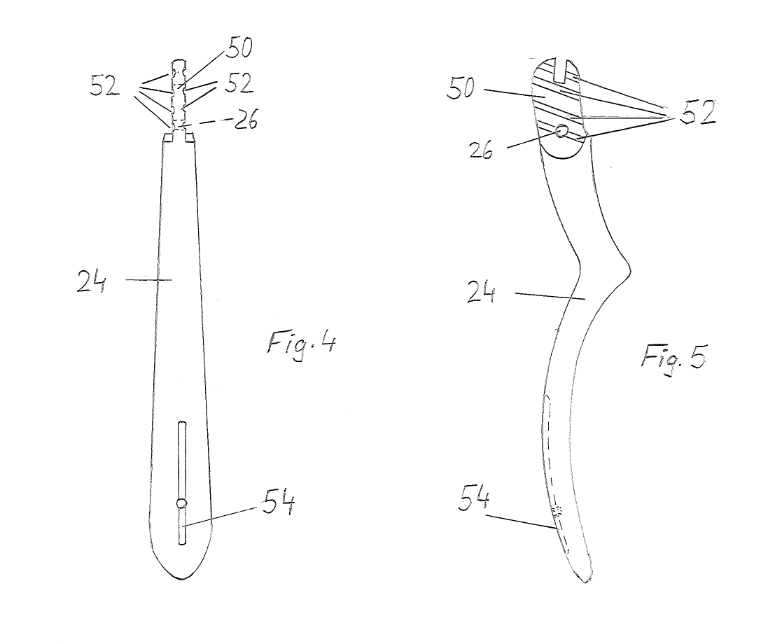

Additional or alternatively further possible ways of rinsing the end region may be provided, as can be seen from FIGS. 3 to 5. For this purpose the tab 50 has one or more rinsing channels 52 in its outer face, preferably in both its outer faces. The rinsing channels 52 are each milled into the outer face of the tab 50 and may for example have a depth of 0.3 mm. The rinsing channels 52 extend approximately transversely with respect to the longitudinal direction of the second handle branch 24 and approximately parallel to the longitudinal direction of the first handle branch 22 when the handle 20 is in its spread-apart position, as can be seen in FIG. 3. An advantageous rinsing of the faces which adjoin one another of the tab 50 of the second handle branch 24 and of the receiving slot of the first handle branch 22 is possible through the rinsing channels 52 without the instrument having to be dismantled.

Additional or alternatively rinsing channels 54 can also be provided in the regions of the fixed first handle branch 22 and the pivotable second handle branch 24 on which the springs 28 abut and are fastened. These rinsing channels 54 extend in the longitudinal direction of the handle branches 22 and 24 over such a length that the rinsing channels 54 lead out with their respective ends beyond the abutting springs 28. As a result rinsing liquid can flow through the rinsing channels 54 below the springs 28 and can clean the contact surfaces of the springs 28.

### LIST OF REFERENCE NUMERALS

10 shaft

12 shaft part

14 slide part

16 punch foot

18 punch cutter blade

20 handle

22 first handle branch

24 second handle branch

26 pivot joint

28 springs

30 sliding surface of 14

32 sliding surface of 12

34 upper face of 14

36 lower face of 12

38 first rinsing slots

40 second rinsing slots

42 recess

44 transverse driver pin

46 rinsing bores of 14

48 rinsing bores of 12

50 tab

52 rinsing channels

54 rinsing channels

1. A surgical sliding shaft instrument, comprising:
a shaft part;
a slide part, which is guided so as to be movable in a longitudinal direction on the shaft part;
a shaft working element disposed on a distal end of the shaft part and a slide working element disposed on a distal end of the slide part;
a proximal handle for reciprocal movement of the shaft part and the slide part, out of a rest position into an operative position;
wherein the shaft part and the slide part abut one another with sliding surfaces;
wherein the slide part is interrupted by first rinsing slots, which extend in the longitudinal direction of the slide part, are distributed above the longitudinal extent of the slide part, and pass through from an upper face of the slide part to the sliding surface thereof,
wherein the shaft part is interrupted by second rinsing slots, which extend in the longitudinal direction of the shaft part, are distributed over the longitudinal extent of the shaft part and pass through from an underside of the shaft part to the sliding surface thereof,
wherein the first rinsing slots of the slide part and the second rinsing slots of the shaft part are constructed and disposed so that in the rest position of the instrument the first rinsing slots and the second rinsing slots adjoin one another alternately in the longitudinal direction.

2. The sliding shaft instrument of claim 1, wherein the first rinsing slots and the second rinsing slots have a same length in the longitudinal direction.

3. The sliding shaft instrument of claim 2, wherein the rinsing slots have a length of approximately 10 to 15 mm.

4. The sliding shaft instrument of claim 1, wherein the first rinsing slots and the second rinsing slots have a same width transversely with respect to the longitudinal direction.

5. The sliding shaft instrument of claim 1, wherein the first rinsing slots and the second rinsing slots are disposed centrally in a transverse direction of the slide part and of the shaft part.

6. The sliding shaft instrument of claim 1, wherein the width of the rinsing slots is approximately a quarter of the slide part width.

7. The sliding shaft instrument of claim 6, wherein the width of the rinsing slots is approximately 0.5 to 1.3 mm.

8. The sliding shaft instrument of claim 1, wherein the rinsing slots of the sliding surface are milled into the slide part and the shaft part respectively.

9. The sliding shaft instrument of claim 1, wherein the handle has a first handle branch rigidly connected to the shaft part and a second handle branch which is pivotably connected to the first handle branch and engages on the slide part, wherein additional rinsing options are provided in an end region in which the handle branches engage in one another and the second handle branch engages on the slide part.

10. The sliding shaft instrument of claim 9, wherein a rinsing bore leads from the outer face of the instrument into the end region.

11. The sliding shaft instrument of claim 9, wherein a rinsing channel is provided in the end region in the surfaces in which the first handle branch and the second handle branch abut one another.

12. The sliding shaft instrument of claim 11, wherein the handle has a first handle branch and a second handle branch which is pivotably connected to the first handle branch, the first handle branch and the second handle being spread apart by springs, wherein the rinsing channels are constructed in regions of the first handle branch and the second handle branch in which the springs abut and are fixed to the first and second handle branches.

13. The sliding shaft instrument of claim 6, wherein the width of the rinsing slots is approximately 1.0 mm.

14. The sliding shaft instrument of claim 1, wherein the first rinsing slots of the slide part and the second rinsing slots of the shaft part are constructed and disposed so that in the rest position of the instrument the first rinsing slots and the second rinsing slots adjoin one another in the longitudinal direction alternately without gaps.

15. A surgical sliding shaft instrument, comprising:
a shaft part with a distal end working element, the shaft part having a shaft part sliding surface and a plurality of longitudinally extending shaft rinsing slots;
a slide part with a distal end working element, the slide part having a slide part sliding surface and a plurality of longitudinally extending slide rinsing slots, the shaft part sliding surface and the slide part sliding surface abutting one another and the slide part movable in a longitudinal direction along the shaft part;
a handle for moving the slide part relative to the shaft part to rest and operative positions; and
wherein the slide rinsing slots are located on the slide part and the shaft rinsing slots are located on the shaft part such that, in the rest position, the slide rinsing slots and the shaft rinsing slots are adjacent to one another in the longitudinal direction.

16. The sliding shaft instrument of claim 15, wherein the slide rinsing slots and the shaft rinsing slots have a same length in the longitudinal direction.

17. The sliding shaft instrument of claim 15, further comprising a rinsing channel provided in the handle proximate to a coupling of the shaft part and the slide part.

18. The sliding shaft instrument of claim 15, wherein the handle further comprises a first handle branch and a second handle branch which is pivotably connected to the first handle branch, wherein a first handle rinsing channel is provided in the first handle branch and a second handle rinsing channel is provided in the second handle branch.

19. The sliding shaft instrument of claim 15, wherein the slide rinsing slots and the shaft rinsing slots do not overlap one another.
